(19) United States
(12) Patent Application Publication
(10) Pub. No.: US 2015/0054850 A1
(43) Pub. Date: Feb. 26, 2015

(54) REHABILITATION DEVICE AND ASSISTIVE DEVICE FOR PHANTOM LIMB PAIN TREATMENT

(71) Applicant: SEIKO EPSON CORPORATION, Tokyo (JP)

(72) Inventor: Hideki TANAKA, Chino-shi (JP)

(21) Appl. No.: 14/449,638

(22) Filed: Aug. 1, 2014

(30) Foreign Application Priority Data
Aug. 22, 2013 (JP) 2013-172039

(51) Int. Cl. G09B 19/00; G06T 7/00; G06T 11/60

(52) U.S. Cl. CPC G09B 19/00; G06T 11/60; G06T 7/0044; A61F 2002/5064
USPC 345/633

(57) ABSTRACT

A rehabilitation device for recovering a function of a paralyzed hand includes: a camera which photographs an image of a mark placed on the paralyzed hand and outputs a photographed image; a position recognition unit which takes input of the photographed image and recognizes a position of the paralyzed hand, using the image of the mark; an image forming unit which outputs a dynamic image in which the paralyzed hand moves; and a head-mounted display which displays the dynamic image superimposed on the paralyzed hand.

### BACKGROUND

1. Technical Field

The present invention relates to a rehabilitation device and an assistive device for phantom limb pain treatment.

2. Related Art

When the neural network in the brain is damaged by cerebral apoplexy, the patient's limbs may become immobile. Methods for rehabilitating such patients are devised. One of these methods is to make the patient think so as to move the paralyzed limb and also show the patient an image of the paralyzed limb moving so that the patient has an illusion that the limb is moving.

A patient having his/her limb lost in an accident or the like may have a pain in that limb. This phenomenon is called phantom limb pain. It is known that a similar method is also effective for such patients. A method is employed in which an image causing an illusion that the lost limb actually exists is shown to the patient. With this method, the lost limb is properly recognized in the patient's brain and the pain disappears or is alleviated.

JP-A-2004-298430 discloses a device that shows a patient an image that looks like his/her paralyzed hand or lost hand is moving. According to this technique, plural magnetic sensors are placed on the patient's body. A predetermined magnetic field is applied to the patient to detect the patient's posture. Then, a dynamic image of the hand is displayed on a display device. At this point, the position, posture and size of the hand in the dynamic image are adjusted so that the patient and the hand in the dynamic image are united together.

The patient views the dynamic image and has an illusion that the hand in the dynamic image is a part of his/her own body. In the case of the patient with a lost hand, as a sense of unity of the hand is re-experienced in the brain, the pain in the hand disappears or is alleviated. In the case of the patient with a paralyzed hand, as the neural network is reconstructed in the brain, the paralysis of the hand is improved.

Rehabilitation of a paralyzed body and treatment for phantom limb pain need to be carried out multiple times. Recovery is faster as the frequency of treatment is higher. Particularly, rehabilitation treatment is effective if carried out early from the onset of the symptom. The device disclosed in JP-A-2004-298430 is a large-sized device that is installed in a particular institution. The patient visits the particular institution, waits for his/her turn, and then receives treatment in the presence of the operator of the device. Therefore, the related-art device does not enable quick and easy treatment. Thus, a simple device with which the patient can receive treatment on his/her own is desired.

### SUMMARY

An advantage of some aspects of the invention is to solve at least a part of the problems described above, and the invention can be implemented as the following forms or application examples.

### Application Example 1

This application example is directed to a rehabilitation device for recovering a function of a paralyzed body part. The rehabilitation device includes: an image photograph unit which photographs an image of a mark placed on the paralyzed body part and outputs a photographed image; a recognition unit which takes input of the photographed image and recognizes a position of the paralyzed body part, using the image of the mark; an image forming unit which outputs a dynamic image in which the paralyzed body part moves; and a display unit which displays the dynamic image superimposed on the paralyzed body part.

According to this application example, a mark is placed on the paralyzed body part. The image photograph unit photographs an image of the mark and outputs the photographed image to the recognition unit. The recognition unit extracts the mark from the photographed image. The recognition unit then recognizes the position of the paralyzed body part, using the mark. The image forming unit outputs a dynamic image in which the paralyzed body part moves, to the display unit.

The patient instructs the paralyzed body part to move in the brain and views the dynamic image in which the paralyzed body part moves. In this case, in the patient's brain, the content of the instruction and the visually received information have similar contents. That is, the patient can have a sense that the paralyzed body part moves as instructed. Then, in the patient's brain, the neural network is recovered so as to transmit the instruction information to the paralyzed body part.

In the rehabilitation device of this application example, the image photograph unit and the recognition unit detect the position of the paralyzed body part, using the mark placed on the paralyzed body part. Therefore, the display unit can display the dynamic image superimposed on the paralyzed body part. Thus, the patient can rehabilitate the paralyzed body part with a simple device. In the related-art device, the patient's posture is detected by a large-sized device and therefore it is difficult for the patient to operate the device on his/her own. In contrast, according to the rehabilitation device of this application example, the paralyzed body part is recognized by a simple device and therefore the patient can operate the rehabilitation device on his/her own to receive rehabilitation treatment.

### Application Example 2

This application example is directed to the rehabilitation device according to the application example described above, wherein the recognition unit recognizes a posture of the paralyzed body part, using the image of the mark, and the image forming unit outputs a dynamic image which moves in the same posture as the posture of the paralyzed body part.

According to this application example, the image photograph unit photographs an image of the mark. The recognition unit recognizes the posture of the paralyzed body part, using the image of the mark. The image forming unit outputs a dynamic image which moves in the same posture as the posture of the paralyzed body part. Therefore, even when the paralyzed body part is twisted, the display unit can display an image corresponding to the twisted body part.

Since the patient is paralyzed at the site which receives rehabilitation treatment, it is difficult for the patient to move the paralyzed body part into a predetermined posture. In the rehabilitation device of this application example, even when the paralyzed body part of the patient is twisted, the dynamic image can be displayed, superimposed on the paralyzed body part. Thus, the patient can easily receive rehabilitation treatment.

### Application Example 3

This application example is directed to the rehabilitation device according to the application example described above, wherein the recognition unit recognizes a distance between the mark and the image photograph unit, using the image of the mark, and the image forming unit outputs a dynamic image in which the paralyzed body part moves, with a size corresponding to the distance.

According to this application example, the recognition unit recognizes the distance between the mark and the image photograph unit, using the image of the mark. The mark appears as a smaller image as it moves away from the image photograph unit. Based on the size of the photographed image of the mark, the distance between the mark and the image photograph unit can be recognized. Then, an image with a size corresponding to the distance is displayed. Thus, viewing the image, the patient can experience a bodily sensation that the paralyzed body part moves.

### Application Example 4

This application example is directed to the rehabilitation device according to the application example described above, wherein the recognition unit recognizes a direction in which the paralyzed body part extends, using the image of the mark, and the image forming unit outputs a dynamic image which moves, facing the same direction as the direction that the paralyzed body part faces.

According to this application example, the recognition unit recognizes the direction in which the mark and the paralyzed body part extend, using the image of the mark. Then, an image is displayed in which the body part extends in the same direction as the direction in which the paralyzed body part extends. Thus, viewing the image, the patient can experience a bodily sensation that the paralyzed body part moves.

### Application Example 5

This application example is directed to the rehabilitation device according to the application example described above, wherein the number of image photograph devices provided in the image photograph unit is one.

According to this application example, the number of image photograph devices provided in the image photograph unit is one. Therefore, the rehabilitation device is simple and can be produced easily.

### Application Example 6

This application example is directed to the rehabilitation device according to the application example described above, wherein the mark is placed in a plural numbers on the paralyzed body part.

According to this application example, the mark is placed in a plural number on the paralyzed body part. When the image photograph unit photographs an image of the paralyzed body part, the paralyzed body part has a site that is photographed in the image and a site that cannot be photographed in the image. Since the plural marks are placed, at least one mark is photographed in the image and therefore the recognition unit can recognize the position of the paralyzed body part.

### Application Example 7

This application example is directed to the rehabilitation device according to the application example described above, which further includes an input unit which designates a speed of the dynamic image in which the paralyzed body part moves, outputted by the image forming unit.

According to this application example, the patient can designate the speed of the dynamic image by operating the input unit. Therefore, the patient can adjust the speed of the dynamic image so that the patient can more easily experience a bodily sensation that the paralyzed body part moves, by viewing the image.

### Application Example 8

This application example is directed to an assistive device for phantom limb pain treatment to reduce pain in a lost body part. The assistive device includes: an image photograph unit which photographs an image of a mark placed on a body part continuing from the lost body part; a recognition unit which recognizes a position of the lost body part, using the mark; an image forming unit which outputs a dynamic image in which the lost body part moves; and a display unit which displays the dynamic image at the position of the lost body part.

According to this application example, a mark is placed on the body part continuing from the lost body part. The image photograph unit photographs an image of the mark and outputs the photographed image to the recognition unit. The recognition unit extracts the mark from the photographed image. The recognition unit then recognizes the position of the lost body part, using the mark. The image forming unit outputs a dynamic image which looks like the lost body part moves at the position of the lost body part, to the display unit.

The patient views the dynamic image in which the lost body part moves. At this point, in the patient’ brain, a sensation that the lost body part moves is experienced. Then, in the patient's brain, the neural network is constructed so that the lost body part is correctly recognized.

In the assistive device for phantom limb pain treatment of this application example, the image photograph unit and the recognition unit detect the position of the lost body part, using the mark placed on the body part continuing to the lost body part. Therefore, the patient can rehabilitate the lost body part with a simple device. In the related-art device, since the posture of the patient is detected by a large-sized device, it is difficult for the patient to operate the device on his/her own. In contrast, in the assistive device for phantom limb pain treatment of this application example, since the lost body part is recognized by a simple device, the patient can operate the assistive device for phantom limb pain treatment on his/her own to receive phantom limb pain treatment.

### BRIEF DESCRIPTION OF THE DRAWINGS

The invention will be described with reference to the accompanying drawings, wherein like numbers reference like elements.

FIG. 1 is a block diagram showing the configuration of a rehabilitation device according to a first embodiment.

FIGS. 2A to 2C are schematic views for explaining marks placed on a hand.

FIG. 3 is a flowchart showing procedures for carrying out rehabilitation treatment.

FIGS. 4A to 4F are schematic views for explaining a rehabilitation treatment method.

FIGS. 5A to 5F are schematic views for explaining the rehabilitation treatment method.

FIGS. 6A to 6G are schematic views for explaining phantom limb pain treatment according to a second embodiment.

FIGS. 7A to 7C show modifications. FIG. 7A is a schematic view of an arm to be treated. FIG. 7B is a schematic view of a foot to be treated. FIG. 7C is a schematic view of a leg to be treated.

### DESCRIPTION OF EXEMPLARY EMBODIMENTS

In the embodiments, characteristic examples of a rehabilitation device and a method for rehabilitation using the rehabilitation device are described with reference to the drawings. Hereinafter, the embodiments are described with reference to the drawings. Since each member in each drawing is shown with a recognizable size in each drawing, each member is shown not to scale.

### First Embodiment

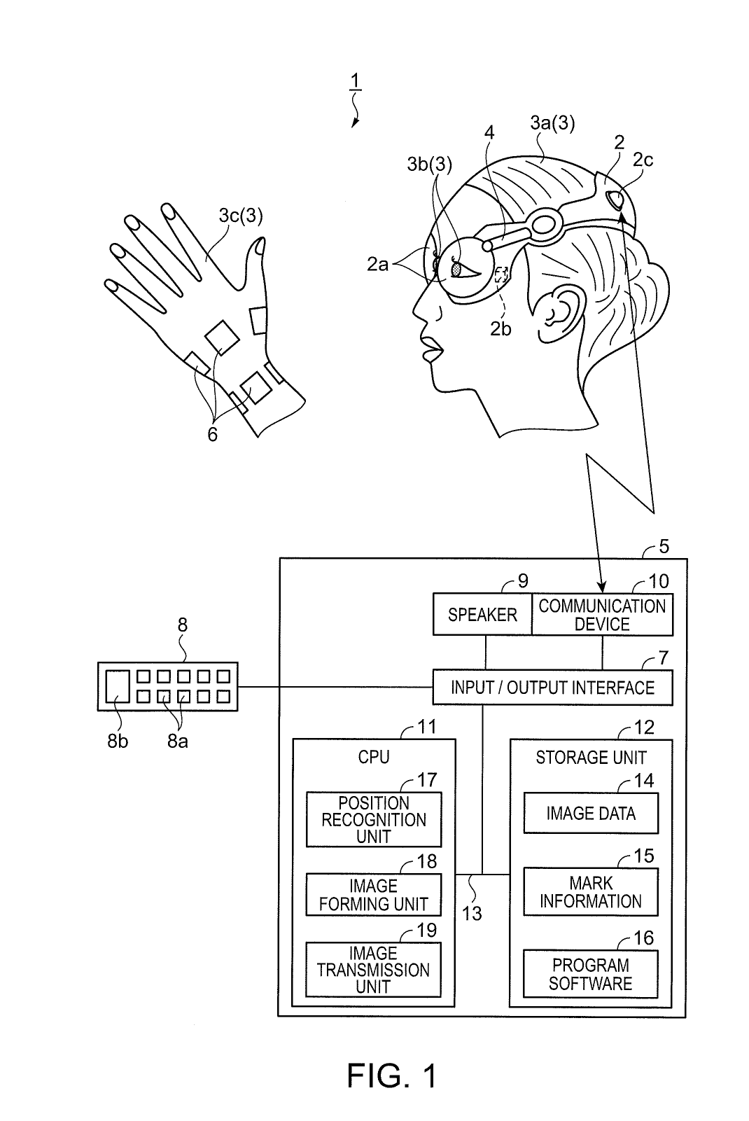

A rehabilitation device according to a first embodiment is described with reference to FIG. 1 to FIGS. 5A to 5F. FIG. 1 is a block diagram showing the configuration of a rehabilitation device. As shown in FIG. 1, a rehabilitation device 1 has a head-mounted display 2 as a display unit. The head-mounted display 2 is placed on a head portion 3a of a patient 3. On the head-mounted display 2, mirror portions 2a are installed in places corresponding to eyes 3b of the patient 3. The head-mounted display 2 has a projection unit 2b. The projection unit 2b emits light to the mirror portions 2a. The light is reflected by the mirror portions 2a and becomes incident on the eyes 3b. The patient 3 can view a dynamic image of a virtual image through the light entering into the eyes 3b. The head-mounted display 2 can show different videos to the right eye and the left eye. Therefore, the head-mounted display 2 can show a stereoscopic image to the patient 3. The mirror portions 2a are non-transmission mirrors.

On the head-mounted display 2, a camera 4 as an image photograph unit and image photograph device is installed. The camera 4 photographs an image within a range that the patient 3 can view. In the camera 4, an objective lens and a CCD (charge coupled device) image photograph element are installed. The camera 4 has an objective lens that can be focused over a long range. The light reflected by an object existing in the field of vision is inputted to the camera 4 via the objective lens, and the light transmitted through the objective lens forms an image on the CCD image photograph element. The image formed on the CCD image photograph element is converted into an electrical signal. Thus, an image of the object existing in the field of vision can be photographed. The camera 4 can use an image photograph tube or CMOS (complementary metal-oxide semiconductor) image sensor instead of the CCD image photograph element.

The head-mounted display 2 has a communication unit 2c. The rehabilitation device 1 has a control device 5. The communication unit 2c communicates with the control device 5 and transmits and receives data to and from the control device 5. The communication unit 2c may employ wireless communication such as communication via radio waves or communication via light, or may employ wired communication. In this embodiment, for example, the communication unit 2c is a device which carries out Bluetooth communication.

The patient 3 has a hand 3c as a paralyzed body part. The patient 3 carries out training to recover the movement of the hand 3c, using the rehabilitation device 1. Plural marks 6 are placed on the hand 3c. The marks 6 are adhesive labels on which a design of a predetermined pattern is drawn. As the adhesive labels are pasted on the hand 3c, the marks 6 can be placed on the hand 3c. The marks 6 are attachable and removable. Also, the marks 6 may be printed on a glove. The patient 3 can wear the glove on the hand 3c and thus place the marks 6 on the hand 3c.

The camera 4 photographs an image of the marks 6 placed on the paralyzed hand 3c and outputs the photographed image to the communication unit 2c. The communication unit 2c transmits the data of the photographed image to the control device 5.

The control device 5 has an input/output interface 7. An input/output terminal 8 as an input unit, a speaker 9 and a communication device 10 are connected to the input/output interface 7. The input/output terminal 8 has input keys 8a and a display panel 8b. The input keys 8a are buttons for the patient 3 to input a content of an instruction when operating the rehabilitation device 1. The display panel 8b is a site where a message to be shown to the patient 3 by the control device 5 is displayed. For example, the control device 5 displays a message which prompts an operation on the display panel 8b, and the patient 3 operates the input keys 8a according to the message. Therefore, the patient 3 can operate the input/output terminal 8 to operate the rehabilitation device 1.

The speaker 9 has the function of communicating a message to the patient 3 as an audio signal. While the patient 3 is receiving rehabilitation treatment, the control device 5 can communicate a message to the patient 3 from the speaker 9 even when the patient 3 is not looking at the display panel 8b.

The communication device 10 is a device which communicates with the communication unit 2c installed on the head-mounted display 2. The communication device 10 and the communication unit 2c communicate the data of the image photographed by the camera 4 and the data of the video emitted from the projection unit 2b, and the like.

The control device 5 also has a CPU 11 (central processing unit) which carries out various kinds of computation processing as a processor, and a storage unit 12 which stores various kinds of information. The input/output interface 7 and the storage unit 12 are connected to the CPU 11 via a data bus 13.

The storage unit 12 conceptually includes a semiconductor memory such as RAM or ROM, and an external storage device such as hard disk or DVD-ROM. Functionally, a storage area for storing image data 14 projected by the projection unit 2b is set. The image data 14 also includes the data of the image photographed by the camera 4. Also, a storage area for storing mark information 15 about the shape of the marks 6, the places where the marks 6 are placed, and the like, is set. Moreover, a storage area for storing program software 16 describing control procedures for the operation of the rehabilitation device 1 is set. Furthermore, a storage area which functions as a work area, temporary file or the like for the CPU 11, and various other storage areas are set.

The CPU 11 is configured to control the rehabilitation device 1 according to the program software 16 stored in the storage unit 12. The CPU 11 has a position recognition unit 17 that is a recognition unit as a specific function realization unit. The position recognition unit 17 takes input of the photographed image. The position recognition unit 17 recognizes the position of the paralyzed hand 3c, using the photographed image of the marks 6. Specifically, the position recognition unit 17 calculates the distance and relative position between the head-mounted display 2 and the marks 6. The position recognition unit 17 then stores the result of the calculation into the storage unit 12 as the mark information 15.

The CPU 11 also has an image forming unit 18. The image forming unit 18 calculates and outputs a dynamic image of a stereoscopic image in which the paralyzed hand 3c moves. The image data 14 of a dynamic image in which the fingers of the hand 3c move to open and close the palm is stored in the storage unit 12. As the mark information 15, the information of the posture and position of the hand 3c calculated by using the photographed image from the camera 4 is stored. The image forming unit 18 has a coordinate transformation function for the dynamic image in which the fingers of the hand 3c move. The image forming unit 18 then performs transformation so that the posture of the hand 3c as viewed from the patient 3 and the posture of the hand in the dynamic image become equal. Next, the image forming unit 18 stores the data of the transformed dynamic image into the storage unit 12 as the image data 14.

The CPU 11 also has an image transmission unit 19. The image transmission unit 19 has the function of transferring the dynamic image data of the image data 14 to the head-mounted display 2. The head-mounted display 2 has a memory for storing the dynamic image data corresponding to a predetermined display time. The image transmission unit 19 then transfers the dynamic image data to the memory of the head-mounted display 2. In the head-mounted display 2, the projection unit 2b projects the dynamic image, using the image data transferred to the memory.

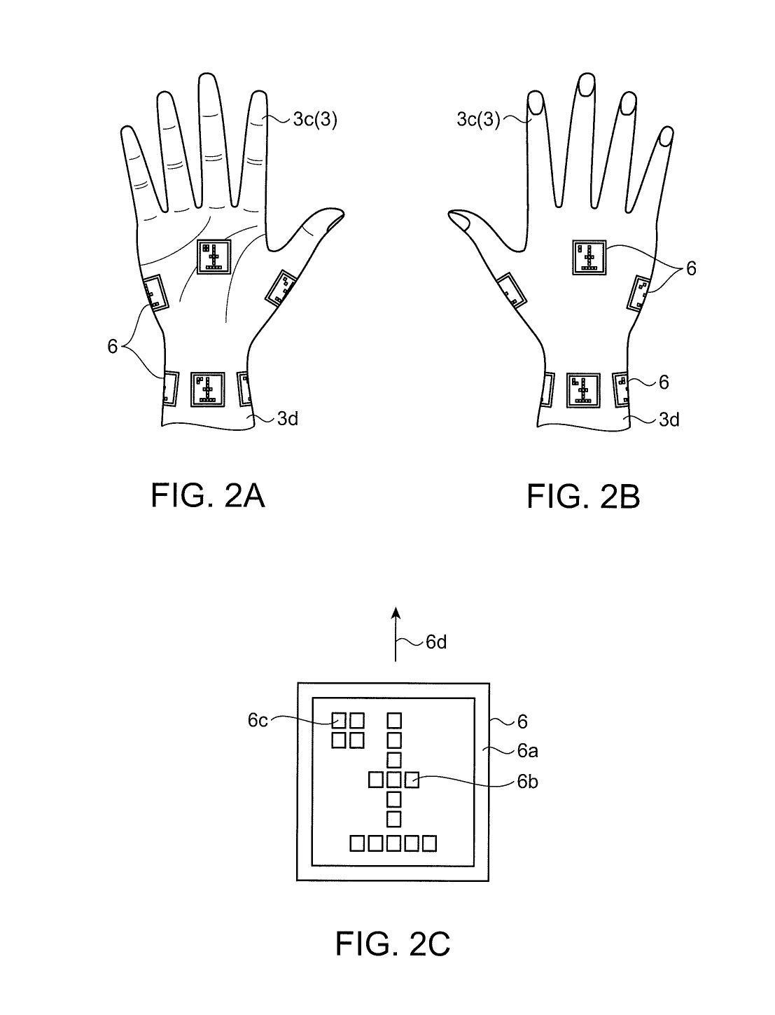

FIGS. 2A to 2B are schematic views for explaining the marks placed on the hand. FIG. 2A shows the state where the marks 6 are placed on the palm side of the hand 3c. FIG. 2B shows the state where the marks 6 are placed on the back side of the hand 3c. FIG. 2C shows the design of the mark 6.

As shown in FIGS. 2A and 2B, plural marks 6 are placed on the hand 3c. Four marks 6 are placed on a wrist 3d. The marks 6 are placed on the palm side, back side, thumb side and little finger side of the wrist 3d. When the wrist 3d is twisted, one or two of the four marks 6 face in the direction of the camera 4. Therefore, even when the patient 3 twists the wrist 3d, the camera 4 can photograph an image of the mark(s) 6.

Moreover, marks 6 are placed also on the palm, the backside, the base of the thumb and the base of the little finger, of the hand 3c. Therefore, even when the patient 3 twists the wrist 3d, the camera 4 can photograph an image of one of the marks 6. By comparing the marks 6 placed on the wrist 3d and the marks 6 placed on the hand 3c, it is possible to recognize whether the wrist joint is twisted or straight.

As shown in FIG. 2C, the mark 6 has the pattern of a frame 6a. The shape of the frame 6a is square. Inside the frame 6a, the pattern of a direction indication drawing 6b that is long in a first direction 6d is provided. The direction indication drawing 6b is a pattern that is narrower on the side of the first direction 6d than on the side opposite to the first direction 6d. The mark 6 is placed on the hand 3c and the wrist 3d in such a way that the first direction 6d indicates the fingertip of the middle finger. Therefore, the directions of the wrist side and the fingertip side of the hand 3c are known from the direction indication drawing 6b. Then, the direction in which the hand 3c extends can be detected.

The mark 6 has an identification drawing 6c. The identification drawing 6c is made up of four quadrilaterals. The identification drawing 6c indicates the place where the mark 6 is placed. Therefore, with the identification drawing 6c, it is possible to identify whether the place of the mark 6 that is photographed in the image is on the side of the wrist 3d, on the palm side, on the back side, or the like. Thus, the position recognition unit 17 can correctly detect the position of the hand 3c.

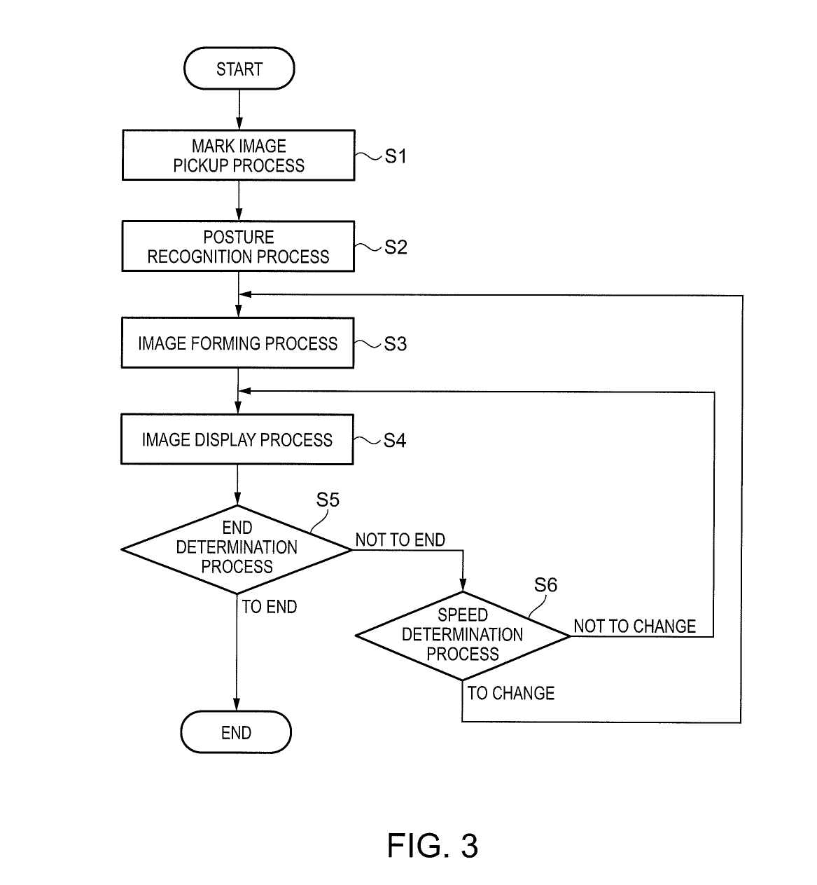

FIG. 3 is a flowchart showing procedures for carrying out rehabilitation treatment. In FIG. 3, Step S1 is equivalent to a mark image photograph process, in which the camera 4 photographs an image of the hand 3c. After the image is photographed by the camera 4, the communication unit 2c transfers the photographed image to the control device 5. In the control device 5, the CPU 11 stores the photographed image into the storage unit 12 as the image data 14. Next, the processing shifts to Step S2. Step S2 is equivalent to a posture recognition process. In this process, the position recognition unit 17 analyzes the photographed image and recognizes the posture of the hand 3c. The position recognition unit 17 recognizes the pattern of the mark 6 and stores the information of the distance between the mark 6 and the camera 4 and the position and direction of the hand 3c, into the storage unit 12 as the mark information 15. Next, the processing shifts to Step S3.

Step S3 is equivalent to an image forming process. In this process, the image forming unit 18 performs coordinate transformation of the dynamic image data, using the mark information 15. The image forming unit 18 performs coordinate transformation of the dynamic image data and thus adjusts the posture of the hand 3c in the dynamic image to the posture of the hand 3c shown in the photographed image. The image forming unit 18 stores the coordinate-transformed dynamic image into the storage unit 12 as the image data 14. Next, the processing shifts to Step S4. Step S4 is equivalent to an image display process. In this process, the image transmission unit 19 transfers the image data 14 of the dynamic image to the head-mounted display 2. Then, the projection unit 2b projects the dynamic image and the patient 3 receives rehabilitation treatment, viewing the dynamic image. Next, the processing shifts to Step S5.

Step S5 is equivalent to an end determination process. In this process, the patient 3 determines whether to continue or end the rehabilitation treatment. If the patient determines not to end but to continue, the processing then shifts to Step S6. If the patient determines to end, the rehabilitation treatment ends. Step S6 is equivalent to a speed determination process. In this process, whether or not the patient changes the speed at which the hand 3c moves in the dynamic image, is determined. If the speed at which the hand 3c moves is to be changed, the processing then shifts to Step S3. If the speed at which the hand 3c moves is not to be changed, the processing then shifts to Step S4. The rehabilitation treatment completes through these processes.

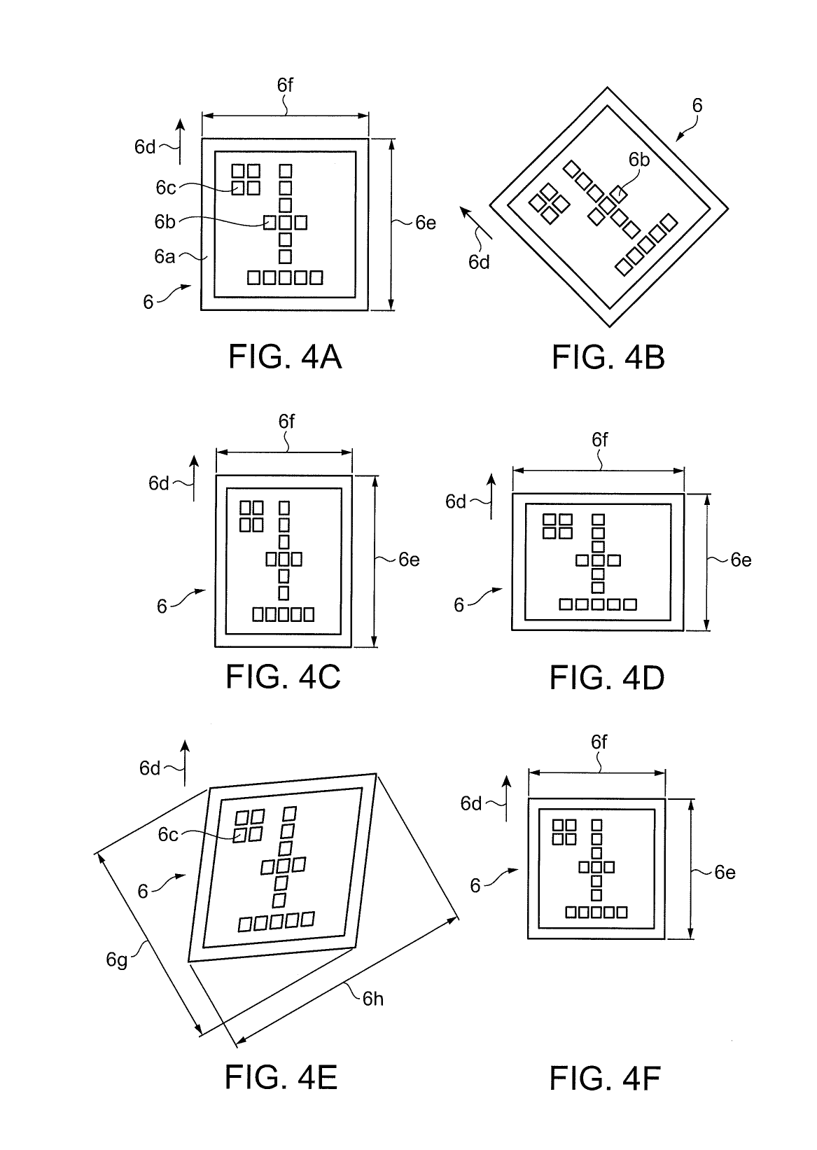

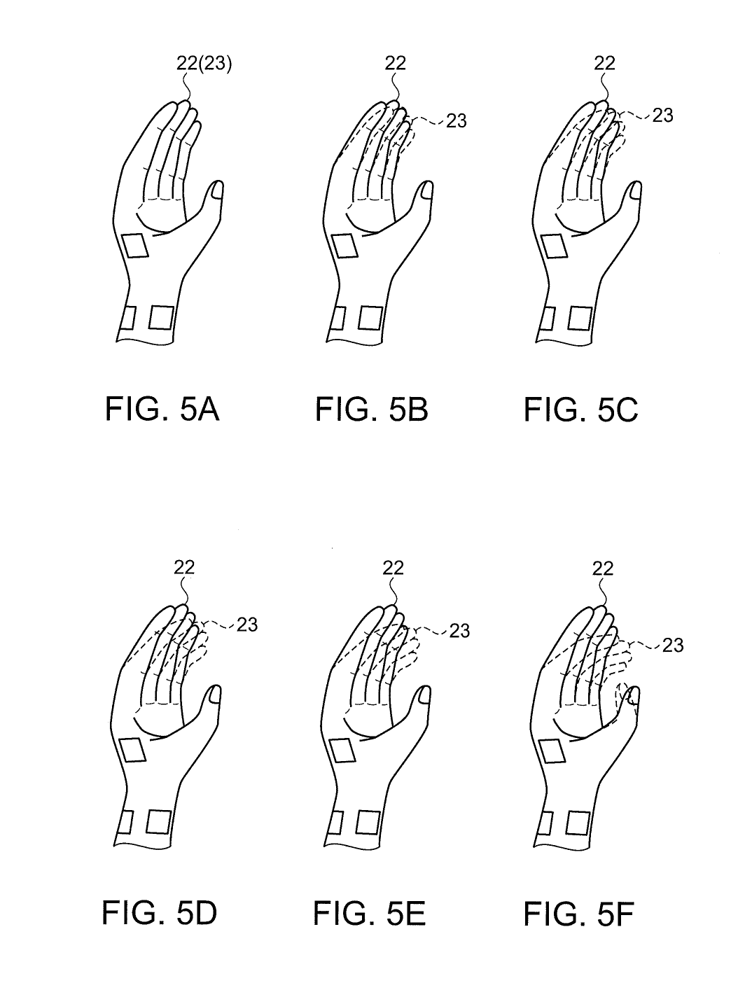

FIGS. 4A to 4F and FIGS. 5A to 5F are schematic views for explaining a rehabilitation treatment method. Next, the rehabilitation treatment method is described in detail, referring to FIGS. 4A to 4F and FIGS. 5A to 5F and in a manner corresponding to the steps shown in FIG. 3. FIGS. 4A to 4F correspond to the mark image photograph process of Step S1 and the posture recognition process of Step S2. In Step S1, the camera 4 photographs an image of the hand 3c, and the position recognition unit 17 extracts the mark 6 from the photographed image. Since plural marks 6 are placed on the hand 3c, the position recognition unit 17 extracts the plural marks 6 and carries out analysis on each of the marks 6. As shown in FIG. 4A, the mark 6 has the identification drawing 6c. The position recognition unit 17 analyzes the identification drawing 6c. Then, based on the identification drawing 6c, the position recognition unit 17 determines which position on the hand 3c the extracted mark 6 is located at.

The position recognition unit 17 also analyzes the direction indication drawing 6b. The position recognition unit 17 analyzes which direction the first direction 6d is, that is, the direction from the wrist toward the fingertip in the hand 3c of the patient 3, in the photographed image. The mark 6 has the square frame 6a. The length in the first direction 6d of the frame 6a of the mark 6 is defined as a first length 6e, and the length in the direction orthogonal to the first direction 6d of the frame 6a is defined as a second length 6f. When the mark 6 is photographed in the image from the front, the first length 6e and the second length 6f are equal.

The direction indication drawing 6b is a pattern elongated in the first direction 6d. The position recognition unit 17 carries out calculation to determine the direction in which the direction indication drawing 6b extends, and thus recognizes the first direction 6d. As shown in FIG. 4B, when the direction indication drawing 6b extends obliquely toward the top left in FIG. 4B, in the image photographed by the camera 4, the position recognition unit 17 recognizes that the first direction 6d is the oblique direction toward the top left in FIG. 4B.

As shown in FIG. 4C, when the second length 6f is shorter than the first length 6e, the face of the mark 6 is a place rotated from the optical axis of the camera 4 about the first direction 6d. As shown in FIG. 4D, when the second length 6f is longer than the first length 6e, the face of the mark 6 is a plane rotated from the optical axis of the camera 4 about the direction orthogonal to the first direction 6d. The position recognition unit 17 estimates the direction in which the mark 6 faces and the angle thereof, using the shape of the contour of the hand 3c and the information of the first length 6e and the second length 6f.

As shown in FIG. 4E, the photographed image of the mark 6 may be rhombic. The length of the diagonal line passing through the identification drawing 6c, of the diagonal lines in the mark 6, is defined as a first diagonal length 6g. The length of the diagonal line that does not pass through the identification drawing 6c, of the diagonal lines in the mark 6, is defined as a second diagonal length 6h. When the second diagonal length 6h is longer than the first diagonal length 6g, the face of the mark 6 is a plane rotated from the optical axis of the camera 4 about the diagonal line indicated by the second diagonal length 6h. The position recognition unit 17 estimates the direction in which the mark 6 faces and the angle thereof, using the shape of the contour of the hand 3c and the information of the first diagonal length 6g and the second diagonal length 6h.

As shown in FIG. 4F, the photographed image of the mark 6 has different sizes depending on the distance from the camera 4. The photographed image of the mark 6 is smaller as the distance from the camera 4 is longer. The position recognition unit 17 calculates the first length 6e and the second length 6f in the image. A distance conversion table that contains data showing the relation between the first length 6e and the second length 6f and the distance from the camera 4 is stored in the storage unit 12. The position recognition unit 17 calculates the distance between the camera 4 and the mark 6, using the first length 6e, the second length 6f and the distance conversion table. Since the number of cameras 4 is one, the rehabilitation device 1 is simple and can be produced easily.

FIGS. 5A to 5F correspond to the image forming process of Step S3 and the image display process of Step S4. As shown in FIGS. 5A to 5F, in Step S3, the image forming unit 18 forms a dynamic image of a stereoscopic image in which the hand 3c moves. The image data 14 in the storage unit 12 includes the dynamic image data of the hand 3c. The image forming unit 18 changes the posture and size of the hand 3c in the dynamic image, based on the dynamic image data and the data of the posture of the hand 3c estimated by the position recognition unit 17. The position recognition unit 17 recognizes the direction in which the paralyzed hand 3c extends, using the image of the mark 6. The image forming unit 18 forms a dynamic image which moves, facing in the same direction as the direction in which the paralyzed hand 3c faces. Then, the image forming unit 18 makes adjustment so that the hand 3c in the dynamic image and the hand 3c photographed in the image by the camera 4 have the same posture and the same size.

In the image photographed by the camera 4, an object at a distance appears small, whereas a nearby object appears large. Similarly, with respect to the shape of the hand 3c in the dynamic image, the image forming unit 18 causes an object at a distance from the camera 4 to appear small, and causes a nearby object to appear large. Thus, the image forming unit 18 can form a perspective image of the hand 3c in the dynamic image.

The solid lines in FIGS. 5A to 5F show a photographed image 22 of the hand 3c photographed by the camera 4. The dotted lines show a simulation image 23 formed by the image forming unit 18. In FIG. 5A, the photographed image 22 and the simulation image 23 are superimposed on each other. In FIG. 5B, the four fingers from the forefinger to the little finger in the simulation image 23 are slightly bent toward the thumb. Then, the movement proceeds in the order of FIG. 5C, FIG. 5D, FIG. 5E and FIG. 5F. In the simulation image 23, the angle at which the four fingers from the forefinger to the little finger are bent increases. When the image shifts from FIG. 5E to FIG. 5F, the thumb bends toward the palm side. Sequence images from FIG. 5A to FIG. 5F are formed and stored as the image data 14 in the storage unit 12.

Next, from FIG. 5F, the movement proceeds in the order of FIG. 5E, FIG. 5D, FIG. 5C, FIG. 5B and FIG. 5A. When shifting from FIG. 5F to FIG. 5A, each finger moves from the bent state to the extended state. Sequence images from FIG. 5F to FIG. 5A are formed and stored as the image data 14 in the storage unit 12.

In Step S4, the image transmission unit 19 transmits the dynamic image data of the image data 14 to the head-mounted display 2. The head-mounted display 2 takes input of the dynamic image data and displays the dynamic image. The patient 3 views the dynamic image and thus can experience a bodily sensation that the hand 3c opens and closes. Then, the patient 3 views the simulation image 23 displayed by the head-mounted display 2 and thus becomes conscious of the opening and closing of the paralyzed hand 3c. Thus, the patient 3 has an illusion that the hand 3c moves, and can receive rehabilitation treatment for the neural system related to the movement of the hand 3c. As the dynamic image displayed by the head-mounted display 2, the images of FIGS. 5A to 5F are sequentially displayed and then the images of FIGS. 5F to 5A are sequentially displayed. Thus, the simulation image 23 is an image in which the fingers are bent and then extended. The simulation image 23 repeats this movement.

In Step S6, the patient 3 determines the opening/closing speed of the hand 3c in the dynamic image. When wanting to change the opening/closing speed of the hand 3c, the patient 3 operates the input/output terminal 8. The CPU 11 determines the content of the operation at the input/output terminal 8. Then, the image transmission unit 19 transmits information of image speed to the head-mounted display 2. The head-mounted display 2 changes the image speed. The input/output terminal 8 serves as a device which designates the speed of the dynamic image in which the hand 3c moves.

In Step S5, when the patient 3 wants to end the rehabilitation treatment, the patient 3 operates the input/output terminal 8 to stop the display of the dynamic image. With these processes, the rehabilitation treatment ends.

As described above, the embodiment has the following effects.

1. According to the embodiment, in the rehabilitation device 1, the camera 4 and the position recognition unit 17 detects the position of the paralyzed hand 3c, using the mark 6 placed on the paralyzed hand 3c. Therefore, the patient can rehabilitate the paralyzed hand 3c with a simple device. In the related-art device, the patient's posture is detected by a large-sized device and therefore it is difficult for the patient to operate the device on his/her own. In contrast, according to the rehabilitation device 1 of the embodiment, the posture of the paralyzed hand 3c is recognized by a simple device and therefore the patient can operate the rehabilitation device 1 on his/her own to receive rehabilitation treatment.

2. According to the embodiment, the camera 4 photographs an image of the mark 6. The position recognition unit 17 recognizes the posture of the paralyzed hand 3c, using the image of the mark 6. The image forming unit 18 forms a dynamic image which moves in the same posture as the posture of the paralyzed hand 3c. Therefore, even when the paralyzed hand 3c is twisted, the head-mounted display 2 can display an image corresponding to the twisted hand 3c.

Since the patient 3 is paralyzed at the site which receives rehabilitation treatment, it is difficult for the patient to move the paralyzed hand 3c into a predetermined posture. In the rehabilitation device 1 of the embodiment, even when the paralyzed hand 3c of the patient is twisted, the dynamic image can be displayed, superimposed on the paralyzed hand 3c. Thus, the patient can easily receive rehabilitation treatment without having to worry about the position and posture of the hand 3c.

3. According to the embodiment, the position recognition unit 17 recognizes the distance between the mark 6 and the camera 4, using the image of the mark 6. Then, an image with a size corresponding to the distance is displayed. Thus, viewing the image, the patient can experience a bodily sensation that the paralyzed hand 3c moves.

4. According to the embodiment, the position recognition unit 17 recognizes the first direction 6d in which the mark 6 and the paralyzed hand 3c extend, using the image of the mark 6. Then, an image is displayed in which the hand 3c extends in the same direction as the direction in which the paralyzed hand 3c extends. Thus, viewing the image, the patient 3 can experience a bodily sensation that the paralyzed hand 3c moves.

5. According to the embodiment, the number of cameras 4 is one. Therefore, the rehabilitation device 1 has a simple configuration and can be produced easily.

6. According to the embodiment, the mark 6 is placed in a plural number on the paralyzed hand 3c. When the camera 4 photographs an image of the paralyzed hand 3c, the paralyzed hand 3c has a site that is photographed in the image and a site that cannot be photographed in the image. Since the plural marks 6 are placed, at least one mark is photographed in the image and therefore the position recognition unit 17 can recognize the position of the paralyzed hand 3c.

7. According to the embodiment, the patient 3 can designate the speed of the dynamic image by operating the input/output terminal 8. Therefore, the patient 3 can more easily experience a bodily sensation that the paralyzed hand 3c moves, by viewing the dynamic image with the speed adjusted.

8. According to the embodiment, the camera 4 is installed on the head-mounted display 2. When the patient 3 looks at the hand 3c, the camera 4 faces in the direction of the hand 3c. Therefore, the camera 4 can photographs a similar image to the hand 3c as viewed from the patient 3. The control device 5 forms a dynamic image based on the image photographed by the camera 4. Therefore, the rehabilitation device 1 can form a dynamic image in which the hand 3c moves in the same posture as the hand 3c as viewed from the patient 3. Thus, the patient 3 can easily experience a bodily sensation that the paralyzed hand 3c moves, by viewing the dynamic image.

### Second Embodiment

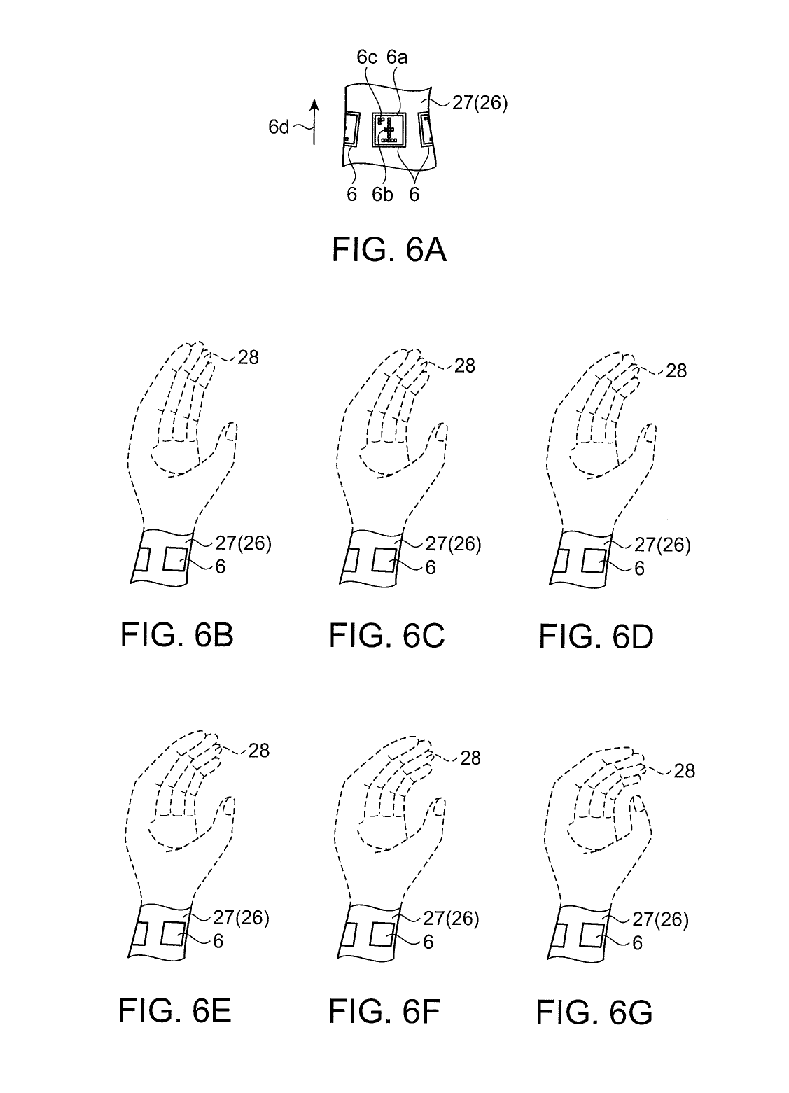

Next, an embodiment of an assistive device for phantom limb pain treatment is described with reference to the schematic views of FIGS. 6A to 6G for explaining phantom limb pain treatment. This embodiment is different from the first embodiment in that an image of the wrist is photographed and then a simulation image of a hand to be connected to the wrist is displayed. The same features as the first embodiment will not be described further in detail.

That is, in this embodiment, the rehabilitation device 1 is used as an assistive device for phantom limb pain treatment. As shown in FIGS. 6A to 6G, a hand connected to a wrist 27 of a patient 26 is lost. Four marks 6 are placed on the wrist 27 at equal spacing in the circumferential direction. The marks 6 are placed on the wrist 27 in the form of labels coated with an adhesive. Also, a wrist band with the marks 6 printed thereon may be worn on the wrist 27. In the marks 6, the frame 6a, the direction indication drawing 6b and the identification drawing 6c are drawn. The rehabilitation device 1 can estimate the place where the lost hand would be located with respect to the wrist 27, using the marks 6.

The camera 4 photographs an image of the wrist 27, and the communication unit 2c transmits the photographed image to the communication device 10. The communication device 10 stores the photographed image in the storage unit 12 as the image data 14. The position recognition unit 17 analyzes the image of the wrist 27 and estimates the position and posture of the lost hand. The image forming unit 18 forms a dynamic image of a simulation image of the hand, based on the data of the estimated position and posture of the hand.

The image data 14 in the storage unit 12 stores data of a basic form of the simulation image of the hand. The image forming unit 18 deforms the simulation image of the hand in such a way that the simulation image of the hand in the basic form connects to the photographed image of the wrist 27. The image transmission unit 19 transmits the image of the wrist 27 and the simulation image of the hand to the head-mounted display 2. The head-mounted display 2 displays the image of the wrist 27 and the simulation image of the hand. The patient 26 receives phantom limb pain treatment, viewing the image of the wrist 27 and the simulation image of the hand.

FIGS. 6B to 6G show a simulation image 28 of the hand formed by the image forming unit 18. In FIG. 6B, the four fingers from the forefinger to the little finger are away from the thumb. As the movement then proceeds in the order of FIG. 6B, FIG. 6C. FIG. 6D, FIG. 6E, FIG. 6F and FIG. 6G, the four fingers from the forefinger to the little finger approach the thumb. Next, as the movement proceeds from FIG. 6G in the order of FIG. 6F, FIG. 6E, FIG. 6D and FIG. 6C, the four fingers from the forefinger to the little finger move away from the thumb. In the dynamic image, the movement of the four fingers from the forefinger to the little finger approaching the thumb and then moving away from the thumb is repeated.

The patient 26 watches the movement of the simulation image 28 connected to the wrist 27. The brain of the patient 26 correctly recognizes that the hand part connected to the wrist 27 is lost. Thus, the occurrence of phantom limb pain is restrained.

As described above, the embodiment has the following effects.

1. According to the embodiment, the marks 6 are placed on the wrist 27 continuing to the lost hand. In the rehabilitation device 1, the camera 4 and the position recognition unit 17 detect the position of the lost hand, using the marks 6. Therefore, the patient 26 can receive phantom limb pain treatment of the lost hand with a simple device. In the related-art device, the posture of the patient 26 is detected by a large-sized device and therefore it is difficult for the patient to operate the device on his/her own. In contrast, the rehabilitation device 1 of this embodiment recognizes the lost body part with a simple device and therefore the patient can operate the device on his/her own to receive phantom limb pain treatment.

The invention is not limited to the above configurations and various changes and improvements can be added by a person with ordinary skills in the art, without departing from the technical scope of the invention. Modifications are described hereinafter.

### Modification 1

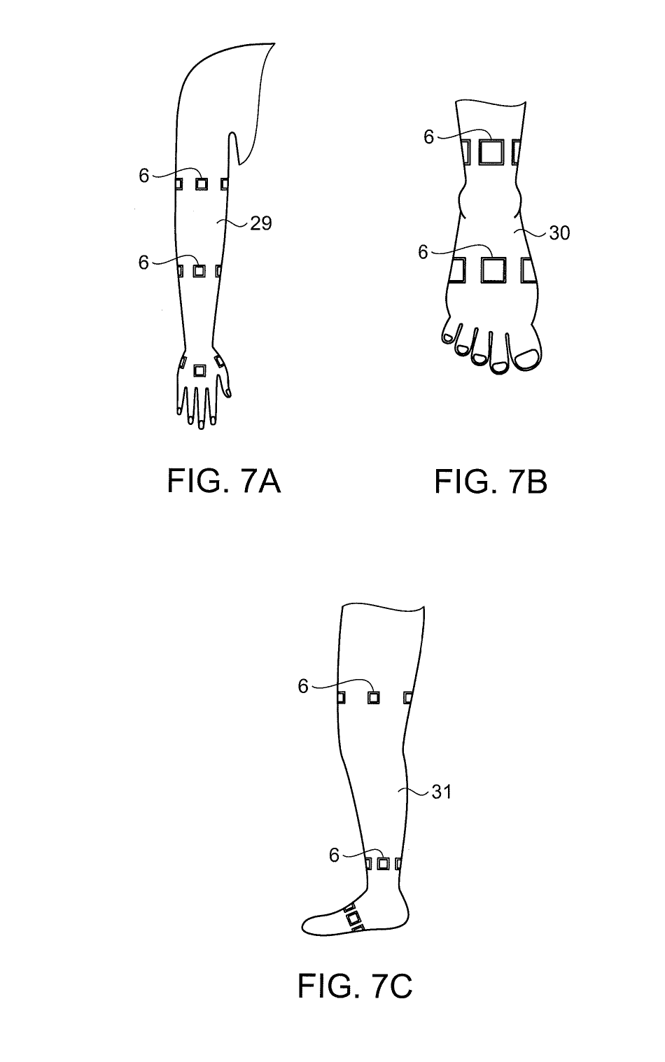

In the first embodiment, the rehabilitation device is used for treatment of the paralyzed hand 3c. The rehabilitation device 1 may also be used for treatment of other body parts than the hand 3c. FIG. 7A is a schematic view of an arm to be treated. As shown in FIG. 7A, plural marks 6 may be placed on an arm 29 and the rehabilitation device 1 may be used for rehabilitation treatment of the arm 29. In this case, in the rehabilitation device 1, a dynamic image in which an arm moves is formed, superimposed on the arm 29, and the dynamic image is displayed on the head-mounted display 2. Thus, the patient can rehabilitate the arm 29 on his/her own.

FIG. 7B is a schematic view of a foot to be treated. As shown in FIG. 7B, plural marks 6 may be placed on a foot and the rehabilitation device 1 may be used for rehabilitation treatment of the foot 30. In this case, in the rehabilitation device 1, a dynamic image in which a foot moves is formed, superimposed on the foot 30, and the dynamic image is displayed on the head-mounted display 2. Thus, the patient can rehabilitate the foot 30 on his/her own.

FIG. 7C is a schematic view of a leg to be treated. As shown in FIG. 7C, plural marks 6 may be placed on a leg 31 and the rehabilitation device 1 may be used for rehabilitation treatment of the leg 31. In this case, in the rehabilitation device 1, a dynamic image in which a leg moves is formed, superimposed on the leg 31, and the dynamic image is displayed on the head-mounted display 2. Thus, the patient can rehabilitate the leg 31 on his/her own.

### Modification 2

In the first embodiment, the image forming unit 18 forms a dynamic image of a stereoscopic image and the head-mounted display 2 displays the stereoscopic image. The image forming unit 18 may form a planar image and the head-mounted display 2 may display the planar image. A planar image has a smaller data volume than a stereoscopic image and therefore can be formed in a short time. Also, the storage capacity of the storage unit 12 can be reduced. Therefore, the rehabilitation device 1 can be produced easily.

### Modification 3

In the first embodiment, the marks 6 are placed on the hand 3c. The pattern of the marks 6 is not limited to the frame 6a, the direction indication drawing 6b and the identification drawing 6c. Other patterns may also be used. For example, circle, ellipse, and polygon may be used. A pattern which is easily recognizable to the position recognition unit 17 may be used.

### Modification 4

In the first embodiment, the single camera 4 is installed on the head-mounted display 2. Two or more cameras 4 may be installed. Then, the distance between the cameras 4 and the mark 6 may be measured using the triangulation method. Also, the distance between the cameras 4 and the marks 6 may be measured using a focusing mechanism. A method that enables easy measurement may be used.

### Modification 5

In the first embodiment, the plural marks 6 are placed on the hand 3c. A single continuous mark may also be placed on the hand 3c. Then, the posture of the hand 3c may be learned, based on the pattern in the place photographed in the image by the camera 4.

### Modification 6

In the first embodiment, the patient can receive rehabilitation treatment on his/her own, using the rehabilitation device 1. An assistant may carry out the rehabilitation treatment. In this case, since the assistant can assist plural patients 3 at the same time, the rehabilitation treatment can be carried out efficiently.

### Modification 7

In the first embodiment, rehabilitation treatment of the hand 3c is carried out using the rehabilitation device 1. Rehabilitation treatment of a finger may also be carried out using the rehabilitation device 1. If a small mark 6 is placed on the finger, the rehabilitation treatment can be carried out as in the first embodiment.

### Modification 8

In the first embodiment, a dynamic image of a movement in which fingers are bent and extended is formed. Dynamic images of other movements may also be formed. For example, a dynamic image in which one finger is extended while the other fingers are bent may be formed. Moreover, movements of rock, paper, and scissors may be employed. By using various dynamic images, the patient 3 can easily continue rehabilitation treatment.

### Modification 9

In the first embodiment, the mirror portions 2a are non-transmission mirrors. The mirror portions 2a may also be a transmission-type. In this case, the image forming unit 18 forms a dynamic image such that the hand 3c viewed through the mirror portions 2a and the hand 3c in the dynamic image are seen as superimposed on each other. Thus, the patient 3 can experience a bodily sensation that the paralyzed hand 3c moves. Moreover, a cover may be provided on the mirror portions 2a to switch between transmission and non-transmission. A technique that enables the patient to easily experience the sensation of the moving hand 3c can be selected.

### Modification 10

In the first embodiment, the head-mounted display 2 displays a dynamic image. This configuration is not limiting and a device which displays a dynamic image between the eyes 3b and the hand 3c of the patient 3 may be arranged. A display device which displays an easily visible dynamic image can be selected. This enables rehabilitation treatment that causes less fatigue.

### Modification 11

In the first embodiment, the photographed image 22 and the simulation image 23 are superimposed on each other and thus displayed. It is also possible to display only the simulation image 23, without displaying the photographed image 22. The patient 3 may also be allowed to select between the display of an image where the photographed image 22 and the simulation image 23 are superimposed on each other and the display of the simulation image 23, by operating the input/output terminal 8. A technique that enables the patient 3 to easily experience the sensation of the moving hand 3c can be selected.

The entire disclosure of Japanese Patent Application No. 2013-172039, filed Aug. 22, 2013 is expressly incorporated by reference herein.

What is claimed is:

1. A rehabilitation device comprising:
an image photograph unit which photographs an image of a mark placed on a paralyzed body part and outputs a photographed image;
a recognition unit which takes input of the photographed image and recognizes a position of the paralyzed body part, using the image of the mark;
an image forming unit which outputs a dynamic image in which the paralyzed body part moves; and
a display unit which displays the dynamic image superimposed on the paralyzed body part.

2. The rehabilitation device according to claim 1, wherein the recognition unit recognizes a posture of the paralyzed body part, using the image of the mark, and
the image forming unit outputs a dynamic image which moves in the same posture as the posture of the paralyzed body part.

3. The rehabilitation device according to claim 1, wherein the recognition unit recognizes a distance between the mark and the image photograph unit, using the image of the mark, and
the image forming unit outputs a dynamic image in which the paralyzed body part moves, with a size corresponding to the distance.

4. The rehabilitation device according to claim 1,
wherein the recognition unit recognizes a direction in which the paralyzed body part extends, using the image of the mark, and
the image forming unit outputs a dynamic image which moves, facing the same direction as the direction that the paralyzed body part faces.

5. The rehabilitation device according to claim 1,
wherein the number of image photograph devices provided in the image photograph unit is one.

6. The rehabilitation device according to claim 1,
wherein the mark is placed in a plural numbers on the paralyzed body part.

7. The rehabilitation device according to claim 1, further comprising an input unit which designates a speed of the dynamic image in which the paralyzed body part moves, outputted by the image forming unit.

8. An assistive device comprising:
an image photograph unit which photographs an image of a mark placed on a body part continuing from a lost body part;
a recognition unit which recognizes a position of the lost body part, using the mark;
an image forming unit which outputs a dynamic image in which the lost body part moves; and
a display unit which displays the dynamic image at the position of the lost body part.
